(19) United States
(12) Patent Application Publication
(10) Pub. No.: US 2026/0232266 A1
(43) Pub. Date: Aug. 13, 2026

(54) AXIAL PHOTODETECTOR ARRAY FOR WEARABLE RING DEVICES

(71) Applicant: Oura Health Oy, Oulu (FI)

(72) Inventors: Tianyu Zeng, Markham (CA); Simon Ratner, San Francisco, CA (US); Nicholas Benjamin Moretto, Reno, NV (US); Chetan Bangalore Chikkamariyappa, Dublin, CA (US); Jose Julio Doval, Escondido, CA (US); Jonathan Sami Watson, San Diego, CA (US); Nicholas Boring, San Diego, CA (US); Gary Watts, Oceanside, CA (US); Avinoam Halpern, Escondido, CA (US); Craig Maurer, San Diego, CA (US); Tommi Heinonen, Oulu (FI)

(21) Appl. No.: 19/534,574

(22) Filed: Feb. 9, 2026

Related U.S. Application Data
(60) Provisional application No. 63/756,724, filed on Feb. 10, 2025.

(51) Int. Cl. A61B 5/00

(52) U.S. Cl. CPC A61B 5/6826; A61B 2562/046

(57) ABSTRACT

Methods, systems, and devices for a wearable ring device are described. In some cases, the wearable ring device may include a ring-shaped housing, where the ring-shaped housing includes an outer surface and an inner surface. The wearable ring device may additionally include a first light emitting component and a second light emitting component arranged in a first radial direction along the inner surface, as well as a first set of light detecting components located at a first position relative to the first light emitting component and the second light emitting component. In such cases, the first set of light detecting components may include at least two light detecting components arranged in an axial direction along the inner surface, where the axial direction is perpendicular to the first radial direction.

### CROSS REFERENCE

The present Application for Patent claims the benefit of U.S. Provisional Patent Application No. 63/756,724 by Zeng et al. entitled “AXIAL PHOTODETECTOR ARRAY FOR WEARABLE RING DEVICES,” filed Feb. 10, 2025, assigned to the assignee hereof, and expressly incorporated herein.

### FIELD OF TECHNOLOGY

The following relates to wearable devices and data processing, including a wearable ring device with an axial photodetector (PD) array.

### BACKGROUND

Some wearable devices may be configured to collect physiological data associated with a user via one or more sensors of the wearable devices. However, in some examples, the one or more sensors used to collect the physiological data, a configuration of the one or more sensors used to collect the physiological data, or both, may limit types of physiological data that may be collected by the wearable devices, accuracy of the physiological data collected by the wearable devices, or both. Thus, the wearable devices may not be capable of providing a complete or accurate view of the user's health.

### BRIEF DESCRIPTION OF THE DRAWINGS

FIG. 1 illustrates an example of a system that supports a wearable ring device with an axial photodetector (PD) array in accordance with aspects of the present disclosure.

FIG. 2 illustrates an example of a system that supports a wearable ring device with an axial PD array in accordance with aspects of the present disclosure.

FIG. 3 shows an example of a wearable ring device that supports an axial PD array in accordance with aspects of the present disclosure.

FIG. 4 shows an example of a wearable ring device that supports multiple sets of electrodes (e.g., and optionally an axial PD array) in accordance with aspects of the present disclosure.

FIG. 5 shows an example of electrode configurations for a wearable ring device with multiple sets of electrodes (e.g., and optionally an axial PD array) in accordance with aspects of the present disclosure.

FIG. 6 shows an example of photodetector PD array configurations for a wearable ring device with an axial PD array in accordance with aspects of the present disclosure.

### DETAILED DESCRIPTION

Some wearable devices may be configured to collect physiological data associated with a user via one or more sensors of the wearable devices. However, in some examples, the one or more sensors used to collect the physiological data, a configuration of the one or more sensors used to collect the physiological data, or both, may limit types of physiological data that may be collected by the wearable devices, accuracy of the physiological data collected by the wearable devices, or both. Thus, the wearable devices may not be capable of providing a complete or accurate view of the user's health.

Accordingly, techniques described herein may relate to a wearable device, such as a wearable ring device, including an array of photodetectors (PDs) arranged axially across a width of the wearable ring device, which may enable the wearable ring device to collect first physiological data with an increased level of accuracy (e.g., as compared to one or more PDs arranged radially across an inner circumference of the wearable ring device), to collect second physiological data based on a comparison of the first physiological data collected via at least two PDs in the array, or both. For example, the wearable ring device may include one or more PDs sets (e.g., sets of PDs), where the one or more PD sets form an array of PDs and where each PD set includes two or more PDs positioned in parallel, axially across the width of the wearable ring device.

As such, the wearable ring device may measure first physiological data, such as photoplethysmogram (PPG) data, via each PD in a PD set and, in some cases, may determine second physiological, such as blood pressure data, based on a comparison between the first physiological data associated with each PD in the set of PDs. For example, a first PD set may include a first PD and a second PD, such that the wearable ring device may measure a first value of the first physiological data via the first PD at a first time and a second value of the first physiological data via the second PD at a second time. Thus, a system associated with the wearable ring device may determine a value of the second physiological data based on a first comparison of (e.g., first difference between) the first value of the first physiological data collected via the first PD and the second value of the first physiological data collected via the second PD, based on a second comparison of (e.g., a second difference between) the first time at which the first value of the first physiological data was collected via the first PD and the second time at which the second value of the first physiological data was collected via the second PD, or both.

Additionally, or alternatively, the system may average values of physiological data associated with each PD in a PD set. For example, the system may average the first value of the first physiological data collected via the first PD and the second value of the first physiological data collected via the second PD to determine an average value of the first physiological data. In such cases, the average value of the first physiological data may be more accurate than the first value of the first physiological data and the second value of the first physiological data independently. Additionally, or alternatively, the system may average values of physiological data associated with multiple PD sets. For example, the system may average a first value of the second physiological data associated with the first PD set with a second value of the second physiological data associated with a second PD set, may average values of the first physiological data associated with multiple PDs across multiple PD sets, or both.

The wearable ring device may additionally, or alternatively, support multiple sets of electrodes, including at least a first set of electrodes (e.g., a first set of one or more electrodes) and a second set of electrodes (e.g., a second set of one or more electrodes), where the first set of electrodes is positioned on (e.g., relative to, at least partially within) an outer surface of the wearable ring device and the second set of electrodes is positioned on an inner surface of the wearable ring device. In such cases, the system may enable a user to touch (e.g., contact) the first set of electrodes (e.g., using a finger on an opposite hand than a hand wearing the wearable ring device) and may measure (e.g., collect) third physiological data, such as electrocardiogram (ECG or EKG) data, bioimpedance (BioZ) data, electrodermal activity (EDA) data, or any combination thereof, based on a signal path (e.g., through a body of the user) created between the first set of electrodes and the second set of electrodes (e.g., based on the user touching the first set of electrodes). In some examples, BioZ data may be indicative of a muscle mass of the user, a body fat percentage of the user, a water content percentage of the user (e.g., hydration level), or any combination thereof. Additionally, or alternatively, the EDA data may be indicative of a galvanic skin response, which may further indicate stress, hot flashes, or the like thereof.

In some cases, to support the multiple sets of electrodes, the wearable ring device may include at least a first portion of the outer surface (e.g., around the first set of electrodes) and a first portion of the inner surface (e.g., around the second set of electrodes) that is conductive. For example, in some cases, the first portion of the outer surface and the first portion of the inner surface may be metallic (e.g., stainless steel, titanium, liquid metal), while a second portion of the outer surface and a second portion of the inner surface may be non-metallic (e.g., to enable signal propagation). In some other examples, the outer surface of the wearable ring device may be made of a non-metal material (e.g., ceramic) and may be coated with a conductive material, where the coating may be less than a threshold thickness to enable signal propagation through the coating. In such cases, a pattern or shape may be laser etched into the coating around (e.g., relative to) each electrode (e.g., of the second set of electrodes) such that each portion of the coating within the pattern or shape (e.g., corresponding to an electrode of the second set of electrodes) may be conductively isolated relative to the rest of the coating.

The wearable ring device may additionally, or alternatively, support a near field communication (NFC) chip (e.g., antenna) capable of communicating NFC signals. In such cases, the NFC signals may enable one or more functions supported by the wearable ring device, such as contactless payment, two factor authentication, locking or unlocking one or more external devices (e.g., door, vehicle, etc.) or the like thereof. In such cases, at least a portion of the outer surface of the wearable ring device may be made of a material that enables propagation of the NFC signals through the outer surface of the wearable ring device.

Aspects of the disclosure are initially described in the context of systems supporting physiological data collection from users via wearable devices. Aspects are then described in the context of wearable ring devices, electrode configurations, and photodetector array configurations. Aspects of the disclosure are further illustrated by and described with reference to apparatus diagrams, system diagrams, and flowcharts that relate to a wearable ring device with an axial PD array.

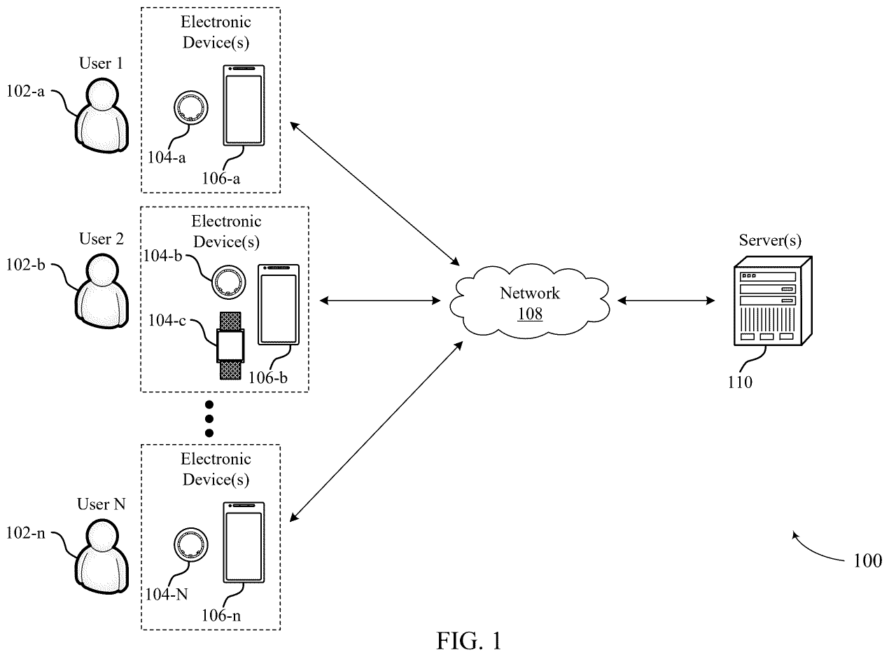

FIG. 1 illustrates an example of a system 100 that supports a wearable ring device with an axial PD array in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols. Moreover, in some cases, the wearable device 104 and the user device 106 may be included within (or make up) the same device. For example, in some cases, the wearable device 104 may be configured to execute an application associated with the wearable device 104, and may be configured to display data via a GUI.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, each ring 104 of the system 100 may include an array of PDs arranged axially across a width of the ring 104, which may enable the ring 104 to collect first physiological data with an increased level of accuracy (e.g., as compared to one or more PDs arranged radially across an inner circumference of the ring 104), to collect second physiological data based on a comparison of the first physiological data collected via at least two PDs in the array, or both. For example, the ring 104 may include one or more PDs sets (e.g., sets of PDs), where the one or more PD sets form an array of PDs and where each PD set includes two or more PDs positioned in parallel, axially across the width of the ring 104. As such, the ring 104 may measure first physiological data, such as PPG data, via each PD in a PD set and, in some cases, may determine second physiological, such as blood pressure data, based on a comparison between the first physiological data associated with each PD in the set of PDs. For example, a first PD set may include a first PD and a second PD, such that the ring 104 may measure a first value of the first physiological data via the first PD at a first time and a second value of the first physiological data via the second PD at a second time. Thus, the system 100 (e.g., respective devices of the system 100) may determine a value of the second physiological data based on a first comparison of (e.g., first difference between) the first value of the first physiological data collected via the first PD and the second value of the first physiological data collected via the second PD, based on a second comparison of (e.g., a second difference between) the first time at which the first value of the first physiological data was collected via the first PD and the second time at which the second value of the first physiological data was collected via the second PD, or both.

Additionally, or alternatively, the system 100 may average values of physiological data associated with each PD in a PD set. For example, the system 100 may average the first value of the first physiological data collected via the first PD and the second value of the first physiological data collected via the second PD to determine an average value of the first physiological data. In such cases, the average value of the first physiological data may be more accurate than the first value of the first physiological data and the second value of the first physiological data independently. Additionally, or alternatively, the system 100 may average values of physiological data associated with multiple PD sets. For example, the system 100 may average a first value of the second physiological data associated with the first PD set with a second value of the second physiological data associated with a second PD set, may average values of the first physiological data associated with multiple PDs across multiple PD sets, or both.

The ring 104 may additionally, or alternatively, support multiple sets of electrodes, including at least a first set of electrodes (e.g., a first set of one or more electrodes) and a second set of electrodes (e.g., a second set of one or more electrodes), where the first set of electrodes is positioned on (e.g., relative to, at least partially within) an outer surface of the ring 104 and the second set of electrodes is positioned on an inner surface of the ring 104. In such cases, the system 100 may enable a user 102 to touch (e.g., contact) the first set of electrodes (e.g., using a finger on an opposite hand than a hand wearing the wearable ring device) and may measure (e.g., collect) third physiological data, such as ECG or EKG data, BioZ data, EDA data, or any combination thereof, based on a signal path (e.g., through a body of the user 102) created between the first set of electrodes and the second set of electrodes (e.g., based on the user 102 touching the first set of electrodes).

The ring 104 may additionally, or alternatively, support an NFC chip (e.g., antenna) capable of communicating NFC signals. In such cases, the NFC signals may enable one or more functions supported by the ring 104, such as contactless payment, two factor authentication, locking or unlocking one or more external devices (e.g., door, vehicle, etc.) or the like thereof. In such cases, at least a portion of the outer surface of the ring 104 may be made of a material that enables propagation of the NFC signals through the outer surface of the ring 104.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

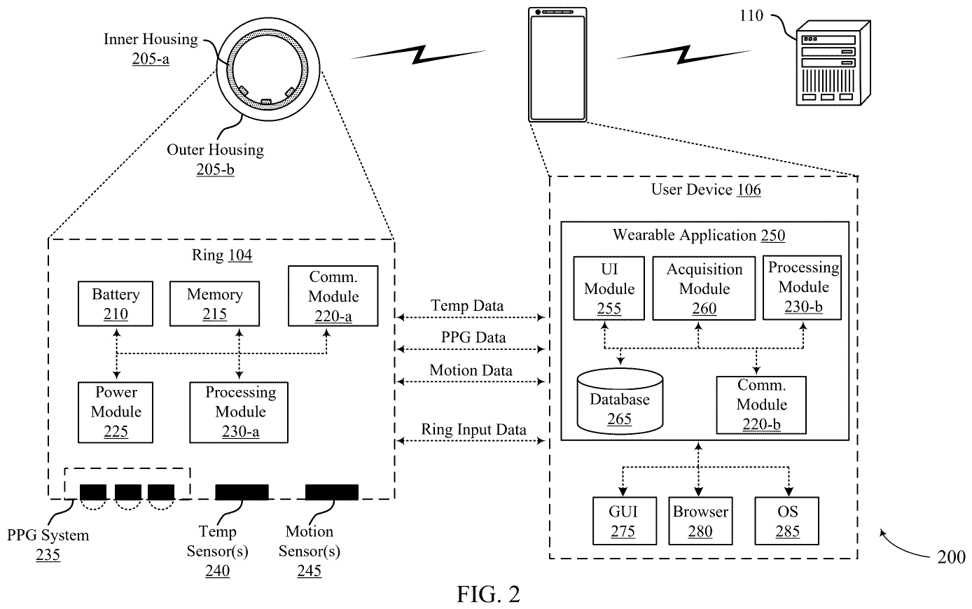

FIG. 2 illustrates an example of a system 200 that supports a wearable ring device with an axial PD array in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, PPG data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a (e.g., including an inner surface of the ring 104) and an outer housing 205-b (e.g., including an outer surface of the ring 104). In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMl160 inertial micro electro-mechanical system (MEMS) sensor that may measure angular rates and accelerations in three perpendicular axes.

The processing module 230-a may sample the motion signals at a sampling rate (e.g., 50Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS) 285, a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

In some cases, the wearable device 104 and the user device 106 may be included within (or make up) the same device. For example, in some cases, the wearable device 104 may be configured to execute the wearable application 250, and may be configured to display data via the GUI 275.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the PPG system 235 of the ring 104 (e.g., of the system 200) may include an array of PDs arranged axially across a width of the ring 104, which may enable the ring 104 to collect first physiological data with an increased level of accuracy (e.g., as compared to one or more PDs arranged radially across an inner circumference of the ring 104), to collect second physiological data based on a comparison of the first physiological data collected via at least two PDs in the array, or both. For example, the ring 104 may include one or more PDs sets (e.g., sets of PDs), where the one or more PD sets form an array of PDs and where each PD set includes two or more PDs positioned in parallel, axially across the width of the ring 104. As such, the ring 104 may measure first physiological data, such as PPG data, via each PD in a PD set and, in some cases, may determine second physiological, such as blood pressure data, based on a comparison between the first physiological data associated with each PD in the set of PDs. For example, a first PD set may include a first PD and a second PD, such that the ring 104 may measure a first value of the first physiological data via the first PD at a first time and a second value of the first physiological data via the second PD at a second time. Thus, the system 200 (e.g., respective devices of the system 200) may determine a value of the second physiological data based on a first comparison of (e.g., first difference between) the first value of the first physiological data collected via the first PD and the second value of the first physiological data collected via the second PD, based on a second comparison of (e.g., a second difference between) the first time at which the first value of the first physiological data was collected via the first PD and the second time at which the second value of the first physiological data was collected via the second PD, or both.

Additionally, or alternatively, the system 200 may average values of physiological data associated with each PD in a PD set (e.g., of the PPG system 235). For example, the system 200 may average the first value of the first physiological data collected via the first PD and the second value of the first physiological data collected via the second PD to determine an average value of the first physiological data. In such cases, the average value of the first physiological data may be more accurate than the first value of the first physiological data and the second value of the first physiological data independently. Additionally, or alternatively, the system 200 may average values of physiological data associated with multiple PD sets. For example, the system 200 may average a first value of the second physiological data associated with the first PD set with a second value of the second physiological data associated with a second PD set, may average values of the first physiological data associated with multiple PDs across multiple PD sets, or both.

The ring 104 may additionally, or alternatively, support multiple sets of electrodes, including at least a first set of electrodes (e.g., a first set of one or more electrodes) and a second set of electrodes (e.g., a second set of one or more electrodes), where the first set of electrodes is positioned on (e.g., relative to, at least partially within) the outer housing 205-b of the ring 104 and the second set of electrodes is positioned on the inner housing 205-a of the ring 104. In such cases, the system 200 may enable a user 102 to touch (e.g., contact) the first set of electrodes (e.g., using a finger on an opposite hand than a hand wearing the wearable ring device) and may measure (e.g., collect) third physiological data, such as ECG or EKG data, BioZ data, EDA data, or any combination thereof, based on a signal path (e.g., through a body of the user 102) created between the first set of electrodes and the second set of electrodes (e.g., based on the user 102 touching the first set of electrodes).

In some cases, to support the multiple sets of electrodes, the ring 104 may include at least a first portion of the outer housing 205-b (e.g., around the first set of electrodes) and a first portion of the inner housing 205-a (e.g., around the second set of electrodes) that is conductive. For example, in some cases, the first portion of the outer housing 205-b and the first portion of the inner housing 205-a may be metallic, while a second portion of the outer housing 205-b and a second portion of the inner housing 205-a may be non-metallic (e.g., to enable signal propagation). In some other examples, the outer housing 205-b of the ring 104 may be made of a non-metal material (e.g., ceramic) and may be coated with a conductive material (e.g., the outer housing 205-b may include the non-metal material and the coating), where the coating may be less than a threshold thickness to enable signal propagation through the coating. In such cases, a pattern or shape may be laser etched into the coating around (e.g., relative to) each electrode (e.g., of the second set of electrodes) such that each portion of the coating within the pattern or shape (e.g., corresponding to an electrode of the second set of electrodes) may be conductively isolated relative to the rest of the coating.

The ring 104 may additionally, or alternatively, support an NFC chip (e.g., associated with, or part of, the communication module 220-a) capable of communicating NFC signals. In such cases, the NFC signals may enable one or more functions supported by the ring 104, such as contactless payment, two factor authentication, locking or unlocking one or more external devices (e.g., door, vehicle, etc.), access gym equipment, or the like thereof. In such cases, at least a portion of the outer surface of the ring 104 may be made of a material that enables propagation of the NFC signals through the outer housing 205-a of the ring 104.

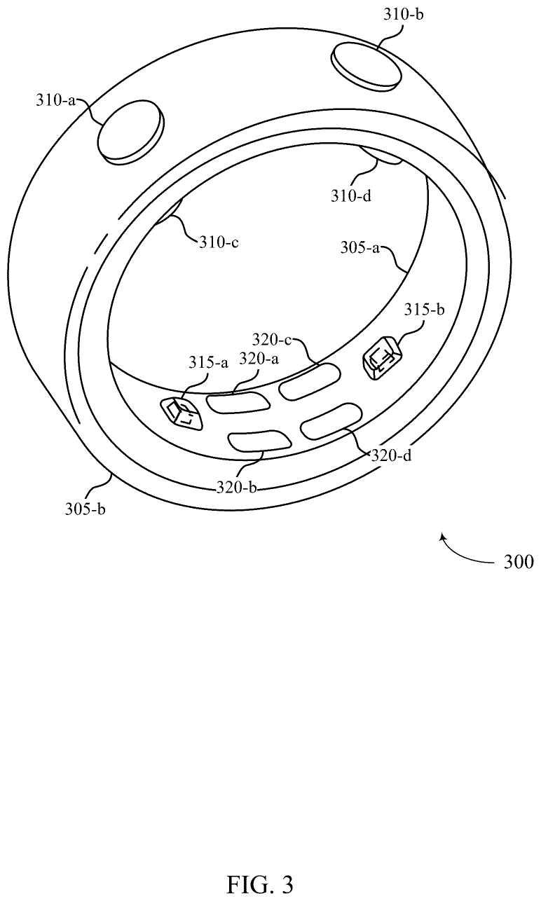

FIG. 3 shows an example of a wearable ring device 300 (e.g., wearable ring device 104, ring 104) that supports an axial PD array in accordance with aspects of the present disclosure.

In some cases, the wearable ring device 300, which may be referred to as a ring 104, may include multiple PDs 320 positioned relative to (e.g., at least partially within, on, embedded into) an inner surface 305-a of the ring 104, where the multiple PDs 320 are arranged axially across a width (e.g., of an inner surface 305-a) of the ring 104. That is, the ring 104 may include one or more PD sets (e.g., one or more sets of PDs 320), where the one or more PD sets form an array of PDs (e.g., axial PD array). In such cases, each PD set may include two or more PDs 320 positioned axially (e.g., and in parallel) across the width of the ring 104. For example, as depicted in FIG. 2, the ring 104 may include a first PD set, including a PD 320-a and a PD 320-b, and a second PD set, including a PD 320-c and a PD 320-b.

In some cases, when the ring 104 includes more than one PD set, as depicted in FIG. 2, the PD sets may be in parallel across an inner circumference (e.g., of the inner surface 305-a) of the ring 104. For example, the PD 320-a may be radially parallel to (e.g., align radially with) the PD 320-c and the PD 320-b may be radially parallel to the PD 320-d. Additionally, or alternatively, any combination of a first distance between the PD 320-a and the PD 320-c, a second distance between the PD 320-b and the PD 320-d, a third distance between the PD 320-a and the PD 320-b, and a fourth distance between the PD 320-c and the PD 320-d may be a same distance. In other words, at least a subset of the PDs 320 may be equally spaced relative to other PDs 320 (e.g., of the ring 104).

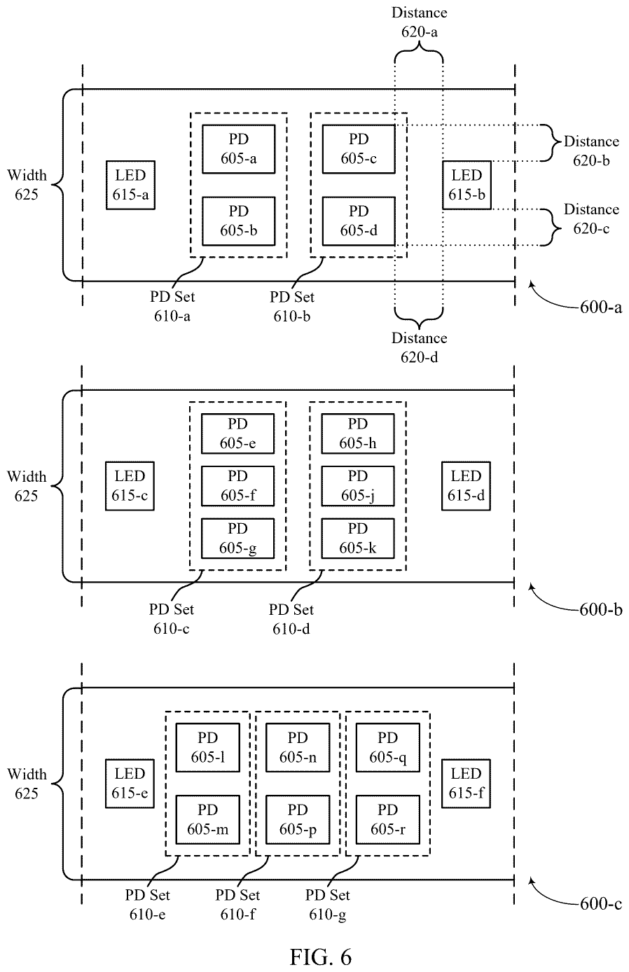

The ring 104 may additionally include one or more light emitting diodes (LEDs) 315 positioned relative to (e.g., at least partially within, on, embedded into, attached to) the inner surface 305-a of the ring 104. For example, as depicted in FIG. 2, the ring 104 may include an LED 315-a and an LED 315-b, where the PDs 320 are positioned between the LEDs 315. Though the PDs 320 are depicted and described as being between the LEDs 315, this is not to be regarded as a limitation of the present disclosure. In this regard, any position of the LEDs 315 relative to the PDs 320 (e.g., and visa-versa) may be supported with regards to the techniques described herein, as described further with reference to FIG. 6. Additionally, or alternatively, the ring 104 may include any quantity of LEDs 315 and any quantity of PDs 320.

As such, the ring 104 may collect physiological data associated with a user 102 wearing the ring 104 (e.g., on a first finger of a first hand of the user 102) based on one or more signals transmitted from at least a subset of the LEDs 315 to at least a subset of the PDs 320. For example, the LED 315-a may be capable of transmitting one or more signals (e.g., one or more optical signals) to each of the PD 320-a (e.g., via a first optical path), the PD 320-b (e.g., via a second optical path), the PD 320-c (e.g., via a third optical path), the PD 320-d (e.g., via a fourth optical path), or any combination thereof. Similarly, the LED 315-b may be capable of transmitting one or more signals (e.g., one or more optical signals) to each of the PD 320-a (e.g., via a fifth optical path), the PD 320-b (e.g., via a sixth optical path), the PD 320-c (e.g., via a seventh optical path), the PD 320-d (e.g., via an eight optical path), or any combination thereof.

In some cases, the ring 104 may collect first physiological data, such as PPG data, associated with the user 102 based on the one or more optical signals and may determine second physiological data, such as blood pressure data, pulse wave velocity (PWV) data, blood viscosity data, arterial stiffness data, or any combination thereof, based on the first physiological data. For example, the ring 104 may measure a first value of the first physiological data via the PD 320-a at a first time (e.g., based on one or more first optical signals from the LED 315-a, the LED 315-b, or both), a second value of the first physiological data via the PD 320-b at a second time (e.g., based on one or more second optical signals from the LED 315-a, the LED 315-b, or both), a third value of the first physiological data via the PD 320-c at a third time (e.g., based on one or more third optical signals from the LED 15-a, the LED 315-b, or both), a fourth value of the first physiological data via the PD 320-d at a fourth time (e.g., based on one or more fourth optical signals from the LED 15-a, the LED 315-b, or both), or any combination thereof. In such cases, any combination of the first time, the second time, the third time, and the fourth time may be a same time.

Additionally, a system associated with the ring 104 (e.g., as described with reference to the system 100, the system 200, or both) may determine (e.g., identify, measure, generate) a value of the second physiological data based on a comparison (e.g., difference) between any combination of the values of the first physiological data, a second comparison between any combination of the times at which the values were collected, or both. For example, the system may determine a first value of the second physiological data based on a first comparison (e.g., a first difference) between the first value of the first physiological data via the PD 320-a and the second value of the first physiological data via the PD 320-b, based on a second comparison (e.g., a second difference) between the first time at which the first value of the first physiological data was collected and the second time at which the second value of the first physiological data was collected, or both. In some cases, the system may display an indication of the first value of the second physiological data to the user 102 (e.g., via a user device 106 associated with the ring 104).

In such cases, the determination of the first value of the second physiological data may be based on a correlation between the first difference, the second difference, or both, and the first value of the second physiological data. That is, the first difference, the second difference, or both, may not be equal to the first value of the second physiological data, but may correlate to the first value of the second physiological data according to a table of values (e.g., correlating differences to values of the second physiological data), using one or more equations, using one or more machine learning (ML) models, or any combination thereof. For example, the system may input any combination of the first value of the first physiological data, the second value of the first physiological data, the first time associated with the first value of the first physiological data, the second time associated with the second value of the first physiological data, first information (e.g., a first identifier, first calibration data) associated with the PD 320-a, second information (e.g., a first identifier, first calibration data) associated with the PD 320-b, the first difference, and the second difference into one or more ML models, such that the one or more ML models may output the first value of the second physiological data.

Additionally, or alternatively, the system may average values of physiological data collected via the multiple PDs 320. For example, the system may average any combination of the first value of the first physiological data collected via the PD 320-a, the second value of the first physiological data collected via the PD 320-b, the third value of the first physiological data collected via the PD 320-c, and the fourth value of the first physiological data collected via the PD 320-d to generate one or more average values of the first physiological data. In such cases, the one or more average values of the first physiological data may be more accurate (e.g., be associated with a higher signal quality, less noise, or both) than the first value of the first physiological data, the second value of the first physiological data, the third value of the first physiological data, and the fourth value of the first physiological data independently. In some cases, the system may display an indication of at least a subset of the one or more average values of the first physiological data to the user 102 (e.g., via the user device 106 associated with the ring 104).

In another example, the system may average the first value of the first physiological data collected via the PD 320-a and the third value of the first physiological data collected via the PD 320-c to generate a first average value of the first physiological data, and may average the second value of the first physiological data collected via the PD 320-b and the fourth value of the first physiological data collected via the PD 320-d to generate a second average value of the first physiological data. Thus, the system may determine (e.g., generate) a value of the second physiological data based on a comparison of the first average value of the first physiological data and the second average value of the first physiological data. In such cases, the first time associated with the first value of the first physiological data may be the same as the third time associated with the third value of the first physiological data and the second time associated with the second value of the first physiological data may be the same as the fourth time associated with the fourth value of the first physiological data.

Additionally, or alternatively, the system may average multiple values of the second physiological data. For example, the system may determine a second value of the second physiological data based on a first comparison between the third value of the first physiological data via the PD 320-c and the fourth value of the first physiological data via the PD 320-d, based on a second comparison between the third time at which the third value of the first physiological data was collected and the fourth time at which the fourth value of the first physiological data was collected, or both. Additionally, the system may average the first value of the second physiological data and the second value of the second physiological data to generate an average value of the second physiological data. In such cases, the average value of the second physiological data may be more accurate than the first value of the second physiological data and the second value of the second physiological data independently.

The ring 104 may additionally, or alternatively, include multiple sets of electrodes 310. For example, the ring 104 may include a first set of electrodes 310, including an electrode 310-a and an electrode 310-b, and a second set of electrodes 310, including an electrode 310-c and an electrode 310-d. Though depicted as two electrodes 310 in each set of electrodes 310, this is not to be regarded as a limitation of the present disclosure. In this regard, the ring 104 may include any quantity of sets of electrodes 310 and each set of electrodes 310 may include any quantity of electrodes 310. For example, though not depicted, the first set of electrodes 310 (e.g., or the second set of electrodes 310) may include a single electrode 310.

In such cases, the first set of electrodes 310 may be positioned relative to (e.g., at least partially within, on, embedded into, attached to) an outer surface 305-b (e.g., an outer housing 305-b) of the ring 104 and the second set of electrodes 310 may be positioned relative to the inner surface 305-a (e.g., inner housing 305-a) of the ring 104. At least part of the outer surface 305-b may define an outer circumference of the ring 104 and at least part of the inner surface 305-a may define an inner circumference of the ring 104. In such cases, the second set of electrodes 310 being positioned relative to the inner surface 305-a may result in the electrode 310-c and the electrode 310-d contacting the first finger of the user 102 (e.g., on the first hand of the user 102) when the user 102 is wearing the ring 104 (e.g., on the first finger).

Thus, in some cases, the ring 104 may measure (e.g., collect) third physiological data associated with the user 102, such as ECG (e.g., or EKG) data, BioZ data, EDA data, pulse arrival time (PAT) (e.g., pulse transit time (PTT)) data, or any combination thereof, when the user 102 contacts (e.g., touches) at least one of the electrode 310-a and the electrode 310-b (e.g., at least one electrode 310 of the first set of electrodes 310). In such cases, the user 102 may contact at least one of the electrode 310-a and the electrode 310-b with another hand of the user 102 (e.g., a second hand of the user 102), a second finger on the other hand of the user 102, an opposite wrist of the user 102 (e.g., an opposite wrist than a wrist associated with the first hand), or the like thereof. When the user 102 contacts at least one of the electrode 310-a and the electrode 310-b, one or more signal paths may be created (e.g., may be available, may be present) between any combination of the electrode 310-a (e.g., if contacted), the electrode 310-b (e.g., if contacted), the electrode 310-c, and the electrode 310-d (e.g., through a body of the user 102), where the third physiological data is based on the one or more signal paths (e.g., collected via transmission of one or more electrical signals via the one or more signal paths). For example, the ring 104 may measure EDA data based on the user 102 contacting at least two of the electrodes 310, may measure ECG data based on the user 102 contacting at least three of the electrodes 310, and may measure BioZ data or PAT data based on the user 102 contacting at least four of the electrodes 310. In some examples, PAT data may utilize, or otherwise be based on, ECG data, which may relate to electrical activity of the heart, and PPG data, which may relate to the movement of blood through one or more tissues of the user, to determine an elapsed time for a pulse to reach different locations of the body of the user 102. In some examples, PAT data may be utilized to determine, or otherwise calculate, a blood pressure of the user 102 (e.g., perform a blood pressure spot check) or one or more additional physiological metrics.

In some cases, the system may prompt the user 102 to contact at least one of the electrode 310-a and the electrode 310-b to enable the ring 104 to collect the third physiological data. In such cases, the user 102 may enable or disable the capability of the system to prompt the user 102. Additionally, or alternatively, the prompting may be based on one or more conditions associated with the user 102, other physiological data associated with the user 102, or both. For example, the system may prompt the user 102 to contact at least one of the electrode 310-a and the electrode 310-b based on the user waking up (e.g., prompts the user in the morning), based on one or more anomalies with other physiological data (e.g., atrial fibrillation (AFib)), such as heart rate data, or the like thereof. Additionally, or alternatively, the system may authenticate an identity of the user 102 based on the third physiological data, such that the system may prompt the user 102 to contact at least one of the electrode 310-a and the electrode 310-b to enable the system to authenticate the user 102.

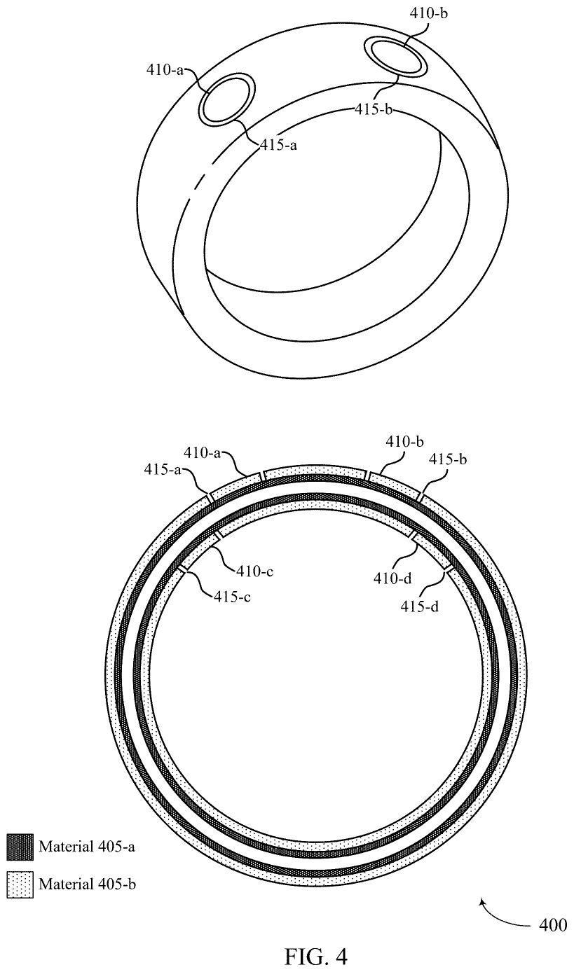

In some cases, to enable creation of the one or more signal paths, the electrodes 310 may be conductively (e.g., electrically) isolated (e.g., insulated) from other parts of the ring 104 (e.g., the rest of the outer housing 305-b and the inner housing 305-a, the LEDs 315, and the PDs 320), as well as each other (e.g., other electrodes 310). For example, in some cases, both the outer housing 305-b and the inner housing 305-a may be made of (e.g., may include) a non-metallic material, while the electrodes 310 may be made of a metallic material (e.g., embedded into the non-metallic material), such that each electrodes 310 may be conductively isolated from the outer housing 305-b, the inner housing 305-a, and other electrodes 310. In some other cases, the ring 104 may be at least partially coated with a material (e.g., a material 405-b), such that the electrodes 310 are conductively isolated from other parts of the ring 104, as described further with reference to FIG. 4.

Additionally, or alternatively, the system may receive one or more user inputs based on the user 102 contacting at least one of the electrode 310-a and the electrode 310-b. For example, the user 102 tap, swipe, squeeze, or press at least one of the electrode 310-a and the electrode 310-b, where the tap, swipe, squeeze, or press correlates to a user input. That is, the system may recognize multiple user inputs, where each user input is associated with a type of contact (e.g., tap, swipe, squeeze, or press), a quantity of electrodes 310 contacted (e.g., the electrode 310-a, the electrode 310-b, or both), an input sequence associated with the contact (e.g., two taps vs. one tap), or any combination thereof. Additionally, or alternatively, the electrodes 310 may support one or more charging mechanisms of the ring 104.

In some cases, the ring 104 may additionally, or alternatively, include one or more NFC chips (e.g., antennas) capable of communicating NFC signaling with one or more external devices. In such cases, the NFC signaling may enable one or more functions supported by the ring 104, such as contactless payment, two factor authentication, locking or unlocking the one or more external devices (e.g., door, vehicle, etc.), accessing (e.g., of pairing the ring 104 with) one or more pieces of gym equipment, or the like thereof. For example, the user 102 may tap the ring 104 on at least a portion of a treadmill, which may automatically log the user 102 into an account associated with the treadmill and may pair the ring 104 with the treadmill, such that the user 102 may view data collected by the ring 104, such as heart rate, via a screen on the treadmill.

In such cases, at least a portion of the outer surface 305-b of the ring 104 (e.g., positioned relative to, or radially above, the NFC chip) may be made of a material that enables propagation of the NFC signals through the outer surface 305-b of the ring 104. For example, the at least portion of the outer surface 305-b (e.g., a window in the outer surface 305-b on top of the ring 104) may be made of a non-metal material (e.g., ceramic material with a physical vapor deposition (PVD) coating) or may be made of a metal material with a thickness less than a threshold thickness, as described with reference to FIG. 4. In some examples, the non-metal material may have similar visual properties as a metal material (e.g., may look like metal).

In some examples, the at least portion of the outer surface 305-b may additionally, or alternatively, be made of a material (e.g., a translucent or transparent material) that enables propagation of ultra-violet (UV) rays through the outer surface 305-b of the ring 104. In such cases, the ring 104 may estimate, or otherwise determine, a level of exposure to UV rays throughout a duration (e.g., a day). For example, the ring 104 may determine a subset of the duration during which the user 102 was exposed to UV rays (e.g., 5 hours of the day). Additionally, or alternatively, the at least portion of the outer surface 305-b may enable propagation of light from beneath the outer surface to outside of the ring 104. For example, the ring 104 may activate an LED positioned beneath (e.g., relative to) the at least portion of the outer surface 305-b to indicate a status (e.g., or state) of the ring 104 to the user 102.

In some examples, the ring 104 may support a modular design. That is, each component of at least a subset of the components of the ring 104, such as the LEDs 315, the PDs 320, the electrodes 310, and the NFC chip, may be associated with a respective hardware module (e.g., with one or more connectors), software module, or both, such that different rings 104 may support different combinations of modules. For example, the ring 104 may be associated with a base module (e.g., hardware and software module) and one or more additional modules may be connected to the base module to support different combinations of features. As an illustrative example, a first module may be associated with the LEDs 315 and the PDs 320, a second module may be associated with the electrodes 310, and a third module may be associated with the NFC chip. Thus, a first ring 104 may support collection of the first physiological data and the second physiological data based on the first ring 104 include the base module and the first module, while a second ring 104 may support collection of the first physiological data, the second physiological data, and the third physiological data based on the first ring 104 include the base module, the first module, and the second module.

Though described in the context of PDs 320 and LEDs 315, this is not to be regarded a limitation of the present disclosure. In this regard, the PDs 320 are merely an exemplary embodiment of a light detecting component and the LEDs 315 are merely an exemplary embodiment of a light emitting component, such that any type of component (e.g., or device) capable of emitting light and any type of component (e.g., or device) capable of receiving light may be considered with regards to the techniques described herein. For example, a light emitting component may include multiple LEDs 315, such as a red LED 315, a green LED 315, and an infrared LED 315.

FIG. 4 shows an example of a wearable ring device 400 (e.g., wearable ring device 104, ring 104) that supports multiple sets of electrodes 410 (e.g., and optionally an axial PD array) in accordance with aspects of the present disclosure.

In some cases, as described with reference to FIG. 3, the ring 104 may include multiple electrodes 410 that are conductively (e.g., electrically) isolated from an outer housing of the ring 104, an inner housing of the ring 104, other electrodes 410, other components (e.g., electrical components) of the ring 104, or any combination thereof. In some cases, to achieve the conductive isolation, the ring 104 may include multiple layers of material 405. For example, as depicted in FIG. 3, the ring 104 may include a first layer of a material 405-b that at least partially surrounds a PCB (e.g., electronic flex assembly) of the ring 104 (e.g., as well as one or more other electrical components), where the material 405-b is a non-conductive material 405 (e.g., a non-conductive ceramic substrate, zirconia).

Additionally, the ring 104 may include a second layer of a material 405-a, that at least partially surrounds the first layer of the material 405-b, where the material 405-a is a conductive material (e.g., conductive ceramic coating, WC-C, Titanium nitride (TiN), chromium nitride (CrN)). In such cases, to conductively isolate the electrodes 410, a portion of the ring 104 surrounding each electrode 410 may not include the material 405-a. For example, an area 415-a around an electrode 410-a, an area 415-b around an electrode 410-b, an area 415-c around an electrode 410-c, and an area 415-d around an electrode 410-d may not include (e.g., may be devoid of) the material 405-a. Thus, the areas 415 may conductively isolate the electrodes 410 from the rest of the coating (e.g., the rest of the second layer).

In some examples, the areas 415 may not include the material 405-a based on the areas 415 being covered (e.g., masked) while the ring 104 is coated (e.g., uniformly) with the material 405-a. In some other cases, the areas 415 may not include the material 405-a based on the material 405-a being etched away (e.g., laser etched, removed) from the areas 415. Additionally, in some cases, other areas 415 of the ring 104 that are not depicted may be covered during coating or etched after coating to support other components of the ring 104 (e.g., such as LEDs, PDs, or both).

In the context of FIG. 4, the electrodes 410 may be formed based on the areas 415 not including the material 405-a. That is, a portion of the coating within (e.g., surrounded by) an area 415, which may be referred to as an island, may include one or more electrical connections between the island (e.g., the material 405-a of the island) and one or more components of the PCB, such that the island may function as an electrode 410 (e.g., bio-electrode 410). In such cases, the one or more electrical connections (e.g., providing contact between the PCB and the islands) may include one or more spring loaded pins, a conductive filled epoxy, a conductive filled pressure sensitive adhesive, or any combination thereof.

Additionally, a thickness of the material 405-a (e.g., of the second layer) may be less than a threshold thickness (e.g., 1-2 micro meters), a resistivity (e.g., surface resistance) of the material 405-a may be less than a threshold resistivity (e.g., 1000 ohms), or both, such that NFC signals (e.g., as well as other signals such as Bluetooth or inductive charging signals) may propagate through the second layer. In other words, the material 405-a (e.g., a conductivity, thickness, or both) may be selected to enable transparency of electromagnetic (EM) radiation through the material 405-a (e.g., through the second layer). In such cases, the EM transparency may be based on source impedance, a wavelength of the EM radiation, a thickness of the material 405-a (e.g., of the second layer), a conductivity of the material 405-a, or any combination thereof.

Though FIG. 3 depicts the ring 104 coated entirely in the material 405-a (e.g., the outer housing and the inner housing form a singular housing component), this is not to be regarded as a limitation of the present disclosure. In this regard, the coating of the material 405-a may be applied to any portion or portions of the ring 104, including, but not limited to, just the outer housing, just the inner housing, a portion of the outer housing, or a portion of the inner housing. As such, in some cases, the ring 104 may support the coating, as well as one or more other techniques described herein, to conductively isolate the electrodes 410.

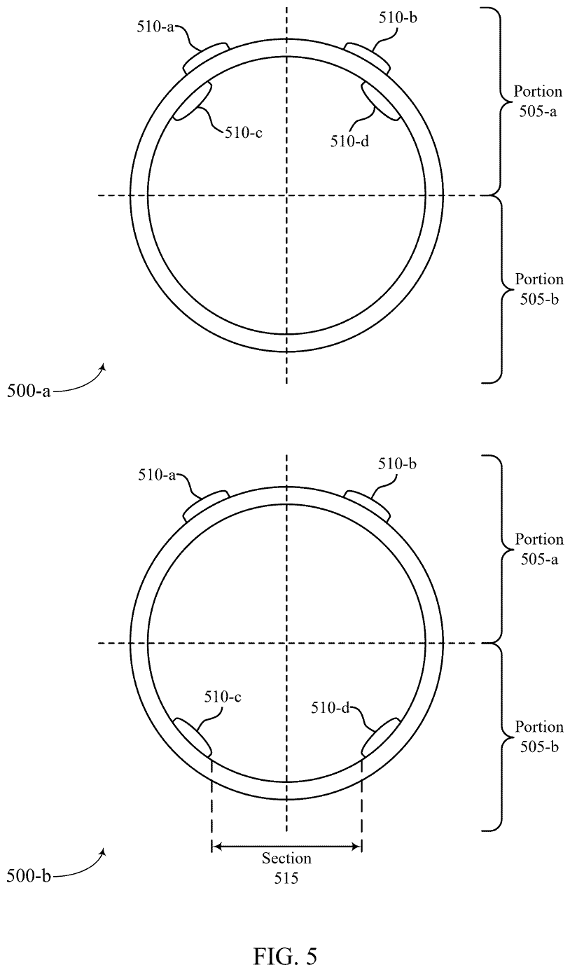

FIG. 5 shows an example of electrode configurations 500 (e.g., an electrode configuration 500-a and an electrode configuration 500-b) for a wearable ring device 104 (e.g., a ring 104) with multiple sets of electrodes 510 (e.g., and optionally an axial PD array) in accordance with aspects of the present disclosure.

In some cases, as described with reference to FIG. 3, the ring 104 may support multiple electrodes 510, including a first set of electrodes 510 (e.g., including an electrode 510-a and an electrode 510-b) positioned relative to (e.g., on) an outer surface of the ring 104 and including a second set of electrodes 510 (e.g., including an electrode 510-b and an electrode 510-c) positioned relative to (e.g., on) an inner surface of the ring 104.

In some cases, as depicted in the electrode configuration 500-a, both sets of electrodes 510 may be positioned within (e.g., relative to) a portion 505-a of the ring 104 (e.g., a portion 505-a of the outer surface and a portion 505-a of the inner surface). In such cases, the portion 505-a may be a top half of the ring 104 that is positioned relative to a top half of a finger of a user 102. That is, while the user 102 is wearing the ring 104 on the finger, the electrode 510-a and the electrode 510-b may face away from a back of a hand of the user 102, while the electrode 510-c and the electrode 510-d may contact at least a portion of a top half of the finger of the user 102 (e.g., a non-palm facing side of the finger). In such cases, one or more LEDs, one or more PDs, or both, as described with reference to FIG. 3, may be positioned within (e.g., relative to) the portion 505-b of the ring 104 (e.g., a portion 505-b of the inner surface).

Additionally, or alternatively, as depicted in the electrode configuration 500-b, the first set of electrodes may be positioned within (e.g., relative to) the portion 505-a of the ring 104 (e.g., the portion 505-a of the outer surface) and the second set of electrodes may be positioned within (e.g., relative to) the portion 505-b of the ring 104 (e.g., the portion 505-b of the inner surface). In such cases, the portion 505-b may be a bottom half of the ring 104 (e.g., opposite the top half). That is, while the user 102 is wearing the ring 104 on the finger, the electrode 510-a and the electrode 510-b may face away from the back of the hand of the user 102, while the electrode 510-c and the electrode 510-d may contact at least a portion of a bottom half of the finger of the user 102 (e.g., a palm facing side of the finger).

In such cases, the one or more LEDs, the one or more PDs, or both, as described with reference to FIG. 3, may be positioned within (e.g., relative to) the portion 505-b of the ring 104 (e.g., the portion 505-b of the inner surface). For example, in some cases, the one or more LEDs, the one or more PDs, or both, may be positioned in (e.g., relative to) the section 515 between the electrode 510-c and the electrode 510-d. In some other cases, at least a subset of the one or more LEDs, at least a subset of the one or more PDs, or both, may be positioned outside of the section 515 (e.g., on either or both sides of the electrode 510-c and the electrode 510-d.

In some cases (e.g., not depicted), the ring 104 may have one or more pads (e.g., soft pads) made of a compressible, or flexible, material on (e.g., attached to) the inner surface of the ring 104 in the portion 505-b. In such cases, the one or more pads may bias, or push, the ring 104 upwards on the finger of the user 102, improving contact between the finger of the user 102 and both the electrode 510-c and the electrode 510-d in the electrode configuration 500-a. The one or more pads may additionally, or alternatively, prevent the ring 104 from rotating around the finger, may support swelling (e.g., and contraction, or changes in size) of the finger (e.g., throughout a day, due to body changes), may enable the ring 104 to support half-sizes of the finger, or any combination thereof. Additionally, or alternatively, the one or more pads may function as electrodes 510.

In some examples, a system associated with the ring 104 may detect an amount, or quantity, of swelling of the finger (e.g., finger swelling data) based on detecting a deflection of the one or more pads. Additionally, or alternatively, the system may detect pre-eclampsia based on BioZ data, blood pressure data, the amount of swelling of the finger, or any combination thereof.

Though depicted in the context of four electrodes 510, this is not to be regarded as a limitation of the present disclosure. In this regard, the ring 104 may include any quantity of electrodes 510 in any electrode configuration 500 or combination of electrode configurations 500, including, but not limited to, the electrode configuration 500-a and the electrode configuration 500-c. For example, the ring 104 may support the electrode configuration 500-a and the electrode configuration 500-b simultaneously (e.g., the ring 104 has six electrodes 510), may support an electrode configuration 500-c (e.g., not depicted) including electrodes 510 on the outer surface of the ring 104 in the portion 505-b of the ring, or both.

FIG. 6 shows an example of PD array configurations 600 (e.g., a PD array configuration 600-a, a PD array configuration 600-b, and a PD array configuration 600-c) for a wearable ring device 104 (e.g., a ring 104) with an axial PD array in accordance with aspects of the present disclosure.

In some cases, as described with reference to FIG. 3, the ring 104 may include one or more PD sets 610, where each PD set 610 includes two or more PDs 605 positioned axially (e.g., and in parallel) across a width 625 of the ring 104. In such cases, the one or more PD sets 610 may be referred to as an axial PD array. In some cases (e.g., not depicted), the ring 104 may include a single PD set 610.

In some other cases, the ring 104 may include multiple PD sets 610. For example, as depicted in the PD array configuration 600-a, the ring 104 may include a PD set 610-a and a PD set 610-b, where each of the PD sets 610 include two PDs 605. That is, the PD set 610-a may include a PD 605-a and a PD 605-b and the PD set 610-b may include a PD 605-c and a PD 605-d. In some examples, both the PD set 610-a and the PD set 610-b may be positioned between a pair of LEDs 615, including an LED 615-a and an LED 615-b. In such cases, the LED 615-a and the LED 615-b may form a radial segment (e.g., of an inner surface of the ring 104) that is perpendicular to both a first axial segment (e.g., of the inner surface of the ring 104) formed by the PD 605-a and the PD 605-b and a second axial segment (e.g., of the inner surface of the ring 104) formed by the PD 605-c and the PD 605-d. In some other examples (e.g., not depicted), the PD set 610-a and the PD set 610-b may be positioned relative to a single LED 315. Additionally, or alternatively, the LED 615-a, the LED 615-b, one or more additional LEDs 615, or any combination thereof, may be positioned between the PD set 610-a and the PD set 610-b. For example, another LED 615 may be positioned between the PD set 610-a and the PD set 610-b.

In another example, as depicted in the PD array configuration 600-b, the ring 104 may include a PD set 610-c and a PD set 610-d, where each of the PD sets 610 include three PDs. That is, the PD set 610-c may include a PD 605-e, a PD 605-f, and a PD 605-g and the PD set 610-d may include a PD 605-h, a PD 605-j, and a PD 605-k. In some cases, both the PD set 610-c and the PD set 610-d may be positioned between a pair of LEDs 615, including an LED 615-c and an LED 615-d. However, similar to the PD array configuration 600-a, other positions of the LEDs 615 relative to the PD sets 610, other quantities of LEDs 615, or both, may be considered in the context of the PD array configuration 600-b.

In another example, as depicted in the PD array configuration 600-c, the ring 104 may include a PD set 610-e, a PD set 610-f, and a PD set 610-g, where each of the PD sets 610 include two PDs. That is, the PD set 610-e may include a PD 605-l and a PD 605-m, the PD set 610-f may include a PD 605-n and a PD 605-p, and the PD set 610-g may include a PD 605-q and a PD 605-r. In some cases, the PD set 610-e, the PD set 610-f, and the PD set 610-g may be positioned between a pair of LEDs 615, including an LED 615-e and an LED 615-f. However, similar to the PD array configuration 600-a, other positions of the LEDs 615 relative to the PD sets 610, other quantities of LEDs 615, or both, may be considered in the context of the PD array configuration 600-c. For example, an additional LED 615 may be positioned between the PD set 610-e and the PD set 610-f, between the PD set 610-f and the PD set 610-g, or both.

In some cases, one or more LEDs 615, one or more PD sets 610, or both, may form an optical chip. For example, the LEDs 615 and the PD sets 610 of the PD array configuration 600-a may form (e.g., create, be associated with) a first optical chip, the LEDs 615 and the PD sets 610 of the PD array configuration 600-b may form a second optical chip, and the LEDs 615 and the PD sets 610 of the PD array configuration 600-b may form a third optical chip. As such, the ring 104 may include any combination of optical chips (e.g., one or more optical chips). For example, the ring 104 may include two of the first optical chips, positioned next to each other radially. In another example, the ring 104 may include the first optical chip and the third optical chip, positioned next to each other radially. Additionally, or alternatively, each PD set 610 associated with the ring 104 may include different quantities of PDs 605. For example, a PD array configuration 600-c (e.g., not depicted) may a PD set 610 with two PDs 605 and a PD set 610 with four PDs 605.

In some cases, as described with reference to FIG. 3, a first value of first physiological data collected via a first PD 605, such as the PD 605-c, based on a first optical signal transmitted from the LED 615-b, may be based on a radial distance 620-a between the PD 605-c and the LED 615-d, an axial distance 620-b between the PD 605-c and the LED 615-d, or both. Similarly, a second value of the first physiological data collected via a second PD 605, such as the PD 605-d, based on a second optical signal (e.g., the same as or different than the first optical signal) transmitted from the LED 615-b, may be based on a radial distance 620-d between the PD 605-d and the LED 615-d, an axial distance 620-c between the PD 605-d and the LED 615-d, or both. As such, a difference between the first value of the first physiological data and the second value of the second physiological data may be based on an axial distance between the PD 605-c and the PD 605-d, a difference between the radial distance 620-a and the radial distance 620-d, a difference between the axial distance 620-b and the axial distance 620-c, or any combination thereof.

In some examples, an axial distance between two neighboring PDs 605 (e.g., two PDs 605 that are next to each other axially, such as the PD 605-c and the PD 605-d) may exceed a threshold distance. That is, the ring 104 may measure (e.g., collect, generate, identify) second physiological data, as described with reference to FIG. 4, based on the axial distance between two neighboring PDs 605 exceeding the threshold distance (e.g., the axial distance may be great enough to identify a difference in first physiological data collected by the neighboring PDs 605).

It should be noted that the methods described above describe possible implementations, and that the operations and the steps may be rearranged or otherwise modified and that other implementations are possible. Furthermore, aspects from two or more of the methods may be combined.

A wearable ring device is described. The wearable ring device may include a housing, the housing comprising an outer housing, wherein the outer housing comprises an outer surface of the wearable ring device, and an inner housing, wherein the inner housing comprises an inner surface of the wearable ring device, a first light emitting component and a second light emitting component arranged in a first radial direction along the inner surface, and a first set of light detecting components located at a first position relative to the first light emitting component and the second light emitting component, the first set of light detecting components comprising at least two light detecting components arranged in an axial direction along the inner surface, wherein the axial direction is perpendicular to the first radial direction.

In some examples of the wearable ring device, the first set of light detecting components comprise a first light detecting component from the at least two light detecting components configured to measure a first value of first physiological data associated with a user based at least in part on receiving a first signal from the first light emitting component, the second light emitting component, or both and a second light detecting component from the at least two light detecting components configured to measure a second value of the first physiological data based at least in part on receiving the first signal from the first light emitting component, the second light emitting component, or both, wherein second physiological data associated with the user may be based at least in part on a first difference between the first value of the first physiological data and the second value of the first physiological data, a second difference between a first time associated with the first value and a second time associated with the second value, or both.

In some examples of the wearable ring device, the second physiological data comprises an average value of the first physiological data.

In some examples of the wearable ring device, the second physiological data comprises a blood pressure data, PWV data, blood viscosity data, arterial stiffness data, or any combination thereof.

In some examples of the wearable ring device, the first difference between the first value and the second value, the second difference between the first time and the second time, or both, may be based at least in part on an axial distance between the first light detecting component and the second light detecting component exceeding a threshold distance.

In some examples of the wearable ring device, the first difference between the first value and the second value, the second difference between the first time and the second time, or both, may be based at least in part on at least one of a first distance between the first light detecting component and the first light emitting component, the second light emitting component, or both, and a second distance between the second light detecting component and the first light emitting component, the second light emitting component, or both.

In some examples of the wearable ring device, a second set of light detecting components located at a second position, different than the first position, relative to the first light emitting component and the second light emitting component, the second set of light detecting components comprising at least two light detecting components arranged in the axial direction along the inner surface.

Some examples of the wearable ring device may further include a third light emitting component arranged in the first radial direction along the inner surface and positioned between the first set of light detecting components and the second set of light detecting components.

In some examples of the wearable ring device, the wearable ring device further comprises a second optical chip located at a fourth position, different than the third position, along the inner surface of the housing, the second optical chip comprising one or more third light emitting components, one or more second sets of light detecting components, or both.

In some examples of the wearable ring device, the first position may be between the first light emitting component and the second light emitting component.

A method is described. The method may include measuring, via a first light detecting component, a first value of first physiological data associated with a user based on receiving a first signal from a first light emitting component, a second light emitting component, or both, where the first light emitting component and a second light emitting component are arranged in a first radial direction along an inner surface of a wearable ring device; measuring, via a second light detecting component, a second value of the first physiological data based on receiving the first signal from the first light emitting component, the second light emitting component, or both, where the first light detecting component and the second light detecting component are arranged in an axial direction along the inner surface of the wearable ring device, and where the axial direction is perpendicular to the first radial direction; and calculating second physiological data associated with the user based on a first difference between the first value of the first physiological data and the second value of the first physiological data, a second difference between a first time associated with the first value and a second time associated with the second value, or both.

In some examples of the method, the second physiological data may include an average value of the first physiological data.

In some examples of the method, the second physiological data may include a blood pressure data, pulse wave velocity data, blood viscosity data, arterial stiffness data, or any combination thereof.

In some examples of the method, the first difference between the first value and the second value, the second difference between the first time and the second time, or both, may be based on an axial distance between the first light detecting component and the second light detecting component exceeding a threshold distance.

In some examples of the method, the first difference between the first value and the second value, the second difference between the first time and the second time, or both, may be based on at least one of a first distance between the first light detecting component and the first light emitting component, the second light emitting component, or both, and a second distance between the second light detecting component and the first light emitting component, the second light emitting component, or both.

In some examples of the method, the wearable ring device may include a second set of light detecting components located relative to the first light emitting component and the second light emitting component, the second set of light detecting components including at least two light detecting components arranged in the axial direction along the inner surface.

In some examples of the method, the first position may be between the first light emitting component and the second light emitting component.

An additional wearable ring device is described. The wearable ring device may include a housing, the housing comprising an outer housing, wherein the outer housing comprises an outer surface of the wearable ring device, and an inner housing, wherein the inner housing comprises an inner surface of the wearable ring device, a first set of electrodes arranged in a first radial direction along the outer surface, the first set of electrodes comprising one or more first electrodes, and a second set of electrodes arranged in a second radial direction along the inner surface, the second set of electrodes comprising two or more second electrodes configured to contact a finger of a user when the wearable ring device may be worn on the finger, wherein the first set of electrodes and the second set of electrodes may be configured to create a signal path through at least a portion of a body of the user when a second finger of the user contacts at least one of the one or more first electrodes associated with the first set of electrodes.

In some examples of the additional wearable ring device, the second set of electrodes may be positioned along a portion of the inner surface.

In some examples of the additional wearable ring device, the portion of the inner surface contacts a side of the finger of the user.

In some examples of the additional wearable ring device, first physiological data measured by the wearable ring device may be based at least in part on transmission of a signal along the signal path.

In some examples of the additional wearable ring device, the first physiological data may include ECG (e.g., or EKG) data, BioZ data, PAT data, EDA data, or any combination thereof.

Some examples of the additional wearable ring device may further include a conductive material coating a first portion of the housing and a plurality of second portions of the housing, wherein the plurality of second portions correspond to respective locations of the one or more first electrodes and the two or more second electrodes, and wherein the plurality of second portions may be conductively isolated from the first portion based at least in part on the plurality of second portions being separated from the first portion.

In some examples of the additional wearable ring device, a plurality of third portions of the housing may be not coated with the conductive material, each third portion from the plurality of third portions surrounds a second portion from the plurality of second portions, and the plurality of second portions being separated from the first portion may be based at least in part on the plurality of third portions.

In some examples of the additional wearable ring device, the first set of electrodes and the second set of electrodes may be conductively isolated from one or more light emitting components, one or more light detecting components, or both.

In some examples of the additional wearable ring device, at least one electrode of the first set of electrodes may be configured to detect a presence of the second finger of the user contacting the first set of electrodes.

In some examples of the additional wearable ring device, one or more user inputs may be based at least in part on the second finger of the user contacting the first set of electrodes.

Some examples of the additional wearable ring device may further include an NFC antenna positioned relative to the housing, the NFC antenna configured to communicate signaling, with one or more external devices, through at least a portion of the housing.

In some examples of the additional wearable ring device, the at least portion of the housing comprises a material that allows propagation of the signaling through the material.

In some examples of the additional wearable ring device, the NFC antenna may be etched into the housing.

Some examples of the additional wearable ring device may further include a conductive material coating at least a first portion of the housing, wherein the NFC antenna may be positioned at least partially within the conductive material.

A method is described. The method may include instructing a user to place a first finger on a first set of electrodes of a wearable ring device, where the first set of electrodes are arranged in a first radial direction along an outer surface of the wearable ring device, the first set of electrodes including one or more first electrodes; and generating a signal for transmission along a signal path through at least a portion of a body of the user in response to the user placing the first finger on the first set of electrodes and based on a second finger of the user contacting a second set of electrodes of the wearable ring device, where the second set of electrodes are arranged in a second radial direction along an inner surface of the wearable ring device and include two or more second electrodes, and where the signal path is between the second set of electrodes and the first set of electrodes.

In some examples, the method may further include measuring first physiological data of the user based on transmission of the signal along the signal path.

In some examples of the method, the first physiological data may include electrocardiogram data, bio impedance data, electrodermal activity data, pulse arrival time, or any combination thereof

In some examples, the method may further include detecting a presence of the second finger of the user contacting the first set of electrodes, where generation of the signal is in response to the detection.

In some examples, the method may further include receiving a user input indicating that the first finger of the user is contacting the at least one electrode of the first set of electrodes, where generation of the signal is in response to the user input

In some examples of the method, a conductive material may coat a first portion of the wearable ring device and a set of second portions of the wearable ring device, where the set of second portions may correspond to respective locations of the one or more first electrodes and the two or more second electrodes, where the set of second portions may be conductively isolated from the first portion based on the set of second portions being separated from the first portion, and where generation of the signal via the signal path is based on the set of second portions being conductively isolated from the first portion.

In some examples of the method, the first set of electrodes and the second set of electrodes may be conductively isolated from one or more light emitting components, one or more light detecting components, or both.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A wearable ring device, comprising:
a housing, the housing comprising:
an outer housing, wherein the outer housing comprises an outer surface of the wearable ring device; and
an inner housing, wherein the inner housing comprises an inner surface of the wearable ring device;
a first light emitting component and a second light emitting component arranged in a first radial direction along the inner surface; and
a first set of light detecting components located at a first position relative to the first light emitting component and the second light emitting component, the first set of light detecting components comprising at least two light detecting components arranged in an axial direction along the inner surface, wherein the axial direction is perpendicular to the first radial direction.

2. The wearable ring device of claim 1, wherein the first set of light detecting components comprise:
a first light detecting component from the at least two light detecting components configured to measure a first value of first physiological data associated with a user based at least in part on receiving a first signal from the first light emitting component, the second light emitting component, or both; and
a second light detecting component from the at least two light detecting components configured to measure a second value of the first physiological data based at least in part on receiving the first signal from the first light emitting component, the second light emitting component, or both, wherein second physiological data associated with the user is based at least in part on a first difference between the first value of the first physiological data and the second value of the first physiological data, a second difference between a first time associated with the first value and a second time associated with the second value, or both.

3. The wearable ring device of claim 2, wherein the second physiological data comprises an average value of the first physiological data.

4. The wearable ring device of claim 2, wherein the second physiological data comprises a blood pressure data, pulse wave velocity data, blood viscosity data, arterial stiffness data, or any combination thereof.

5. The wearable ring device of claim 2, wherein the first difference between the first value and the second value, the second difference between the first time and the second time, or both, are based at least in part on an axial distance between the first light detecting component and the second light detecting component exceeding a threshold distance.

6. The wearable ring device of claim 2, wherein the first difference between the first value and the second value, the second difference between the first time and the second time, or both, are based at least in part on at least one of a first distance between the first light detecting component and the first light emitting component, the second light emitting component, or both, and a second distance between the second light detecting component and the first light emitting component, the second light emitting component, or both.

7. The wearable ring device of claim 1, further comprising:
a second set of light detecting components located at a second position, different than the first position, relative to the first light emitting component and the second light emitting component, the second set of light detecting components comprising at least two light detecting components arranged in the axial direction along the inner surface.

8. The wearable ring device of claim 7, further comprising:
a third light emitting component arranged in the first radial direction along the inner surface and positioned between the first set of light detecting components and the second set of light detecting components.

9. The wearable ring device of claim 1, wherein the first light emitting component, the second light emitting component, and the first set of light detecting components are associated with a first optical chip located at a third position along the inner surface of the housing, and wherein the wearable ring device further comprises:
a second optical chip located at a fourth position, different than the third position, along the inner surface of the housing, the second optical chip comprising one or more third light emitting components, one or more second sets of light detecting components, or both.

10. The wearable ring device of claim 1, wherein the first position is between the first light emitting component and the second light emitting component.

11. A method, comprising:
measuring, via a first light detecting component, a first value of first physiological data associated with a user based at least in part on receiving a first signal from a first light emitting component, a second light emitting component, or both, wherein the first light emitting component and the second light emitting component are arranged in a first radial direction along an inner surface of a wearable ring device;
measuring, via a second light detecting component, a second value of the first physiological data based at least in part on receiving the first signal from the first light emitting component, the second light emitting component, or both, wherein the first light detecting component and the second light detecting component are arranged in an axial direction along the inner surface of the wearable ring device, wherein the axial direction is perpendicular to the first radial direction; and
calculating second physiological data associated with the user based at least in part on a first difference between the first value of the first physiological data and the second value of the first physiological data, a second difference between a first time associated with the first value and a second time associated with the second value, or both.

12. The method of claim 11, wherein the second physiological data comprises an average value of the first physiological data.

13. The method of claim 11, wherein the second physiological data comprises a blood pressure data, pulse wave velocity data, blood viscosity data, arterial stiffness data, or any combination thereof.

14. The method of claim 11, wherein the first difference between the first value and the second value, the second difference between the first time and the second time, or both, are based at least in part on an axial distance between the first light detecting component and the second light detecting component exceeding a threshold distance.

15. The method of claim 11, wherein the first difference between the first value and the second value, the second difference between the first time and the second time, or both, are based at least in part on at least one of a first distance between the first light detecting component and the first light emitting component, the second light emitting component, or both, and a second distance between the second light detecting component and the first light emitting component, the second light emitting component, or both.

16. The method of claim 11, wherein the wearable ring device comprises a second set of light detecting components located relative to the first light emitting component and the second light emitting component, the second set of light detecting components comprising at least two light detecting components arranged in the axial direction along the inner surface.

17. The method of claim 11, wherein the first light detecting component is between the first light emitting component and the second light emitting component.
